(19) United States
(12) Patent Application Publication
(10) Pub. No.: US 2011/0282254 A1
(43) Pub. Date: Nov. 17, 2011

(54) DECORATIVE CAST COVER

(76) Inventor: Catherine M. Gadlage, Duluth, GA (US)

(21) Appl. No.: 13/105,452

(22) Filed: May 11, 2011

Related U.S. Application Data
(60) Provisional application No. 61/333,868, filed on May 12, 2010.

(51) Int. Cl. A61F 5/01

(52) U.S. Cl. 602/3

(57) ABSTRACT

A cast cover includes a sleeve configured to be disposed over a cast; an aperture configured to receive the cast when pulling the sleeve over the cast; and a comfort band generally disposed about the aperture, wherein the comfort band is configured to be in physical communication with the skin of a cast wearer and covers an edge of the cast.

### CROSS REFERENCE TO RELATED APPLICATION

This application claims benefit to U.S. Provisional Patent Application Ser. No. 61/333,868 filed May 12, 2010, which is fully incorporated herein by reference.

### BACKGROUND

The present disclosure relates to medical devices such as casts. More specifically, the present disclosure relates to a protective cover for use with casts and similar devices to decoratively conceal the cast and inhibit damage to clothing and the like commonly caused by the cast.

Casts, splints, or other devices are commonly used to stabilize an injury site and aid in the healing of broken bones, more severe sprains, etc. As these injuries are not uncommon, estimated to be approximately 6.5 million per year in the USA, with most requiring a second cast or splint, many individuals are required to wear a cast or other device at some time. These devices are used for a substantial period of time. Casts, for example, are typically worn for a period of four to six weeks. Historically, casts have been made of plaster. Most current casts, however are formed of fiberglass mesh as this material better allows the skin to breathe and is more resistant to damage, water, etc.

It is not uncommon for a person wearing such a cast or other such device to inadvertently damage (e.g., dent, scratch, tear, pull, and the like) other items such as furniture, clothing, automobiles, etc. The mesh casts obviously need to be hard so as to prevent accidental movement of a broken bone or injured body part, but the material generally has a coarse surface and the cast can have many rough and/or sharp edges. These edges and rough surface are sufficiently sharp and abrasive to damage many common items such as furniture, clothing, etc. A person with an arm cast may, for example, brush the cast against their clothing, snagging, tearing or damaging the item of clothing. The person may even harm themselves or other persons around them, such as by bruising, scraping, or even cutting them as a result of contact between the person and the cast.

Another problem with casts is that they get dirty and are difficult to wash. A person wearing a cast must still perform many daily tasks such as eating, working, cleaning, and the like. Casts quickly get dirty during these everyday activities. The individual wearing the cast is instructed to keep the cast dry, preventing cleaning of the cast. However, the small holes of the mesh readily accept and hold dirt, food, etc. A further limitation of wearing a cast is the visibility of the cast itself While a minor inconvenience for some, others having a cast while at work or social engagements may view them as visually obtrusive and prefer they be concealed.

The invention is further illustrated by the following drawings, detailed description, and examples.

### BRIEF SUMMARY OF THE INVENTION

In one embodiment, a cast cover includes a sleeve configured to be disposed over a cast; an aperture configured to receive the cast when pulling the sleeve over the cast; and a comfort band generally disposed about the aperture, wherein the comfort band is configured to be in physical communication with the skin of a cast wearer and covers an edge of the cast.

In another embodiment, a cast cover comprises a sleeve formed of a first material and configured to be disposed over a cast; an aperture configured to receive the cast when pulling the sleeve over the cast; and at least one comfort band formed of a second material disposed about the aperture, wherein the comfort band is configured to be in physical communication with the skin of a cast wearer and covers an edge of the cast.

A method of making a cast cover comprises providing a sleeve configured to be disposed about a cast, wherein the sleeve comprises at least one aperture; positioning a comfort band about the at least one aperture, wherein the comfort band is configured to be in physical communication with the skin of a cast wearer and covers an edge of the cast, and wherein forming the comfort band, comprises extending a knit of the sleeve into a first layer of the comfort band; making a turn at an edge of the comfort band; knitting a second layer formed by the turn o the first layer; and knitting the first and the second layer together on an inside surface of the cast cover near the aperture.

These and other features and advantages of the embodiments of the invention will be more fully understood from the following detailed description of the invention taken together with the accompanying drawings. It is noted that the scope of the claims is defined by the recitations therein and not by the specific discussion of features and advantages set forth in the present description

### BRIEF DESCRIPTION OF DRAWINGS

Referring now to the exemplary drawings wherein like elements are numbered alike in the several figures:

FIG. 1 shows a perspective view of a common arm/wrist cast;

FIG. 2 shows a perspective view of an exemplary embodiment of cast cover covering the cast of FIG. 1;

FIG. 3 shows a perspective view of another embodiment of a cast cover comprising a pocket;

FIG. 4 shows a perspective view of another embodiment of a cast cover covering a full arm/elbow cast;

FIG. 5 shows a perspective view of another embodiment of a cast cover covering a lower leg/ankle cast; and

FIG. 6 shows a perspective view of another embodiment of a cast cover covering a full leg cast.

### DETAILED DESCRIPTION

Disclosed herein are decorative cast covers configured to visually conceal the cast and protect it from dirt, etc., while inhibiting damage caused by the cast, such as to clothing and the like. Generally, a decorative cast cover includes a comfortable sleeve configured to surround the cast. The sleeve further includes an aperture configured to receive the cast. The sleeve can further comprise one or more additional apertures configured to receive one or more appendages, such as a hand, a foot, fingers thumb, and the like. The cast cover further includes one or more comfort bands disposed at the edges of the sleeve, generally in the location of the apertures. The comfort bands, as will be described in more detail below, are configured to be in physical communication with the skin of the cast wearer, thereby covering the cast edges and providing a soft, comfortable feel to the wearer while also holding the cast cover in place.

It will be appreciated that the cast covers described herein can be designed to fit any type, size, shape, and the like of a medical cast. For example, cast covers described herein can cover a typical forearm cast, a full arm cast, a lower leg cast, a full leg cast, and the like. While some of these cast covers and cast types are shown in the figures herein, it is to be understood that the cast covers can be designed for suitable use with virtually any cast currently used to protect injured body parts today. Likewise, the cast covers can be used for children's cast as well as adults, and can even be used for casts on animals, such as domestic household pets.

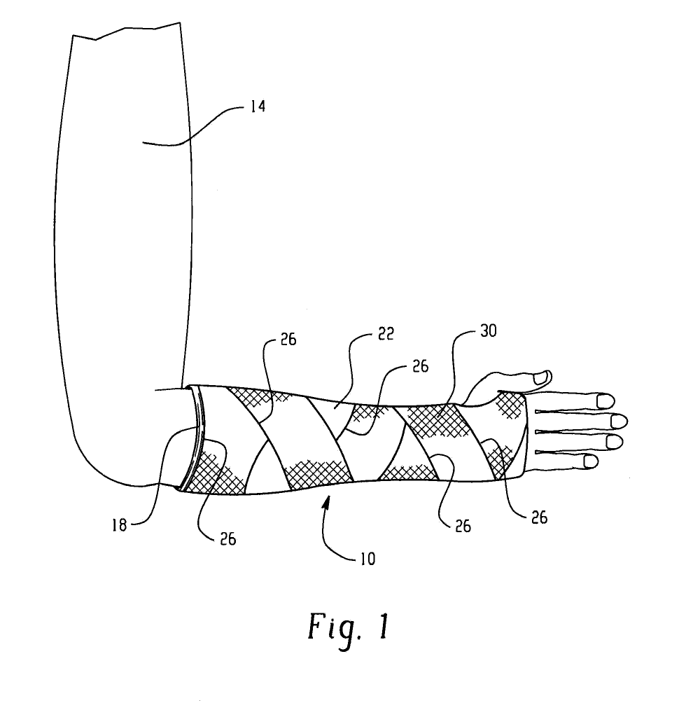

Turning to FIG. 1, a perspective view of a current medical cast is shown. The cast 10 is placed on the arm 14 of an individual. The cast 10 is formed from a padding 18 and multiple wrapped layers of a mesh material 22. The mesh material 22 is bonded together and hardened to form a durable cast 10. The process of making a cast 10 results not only in a coarse mesh surface (shown at 30), but in numerous exposed edges 26 of the mesh material which edges represent the underlying anatomic contours of the stabilized and protected appendage.

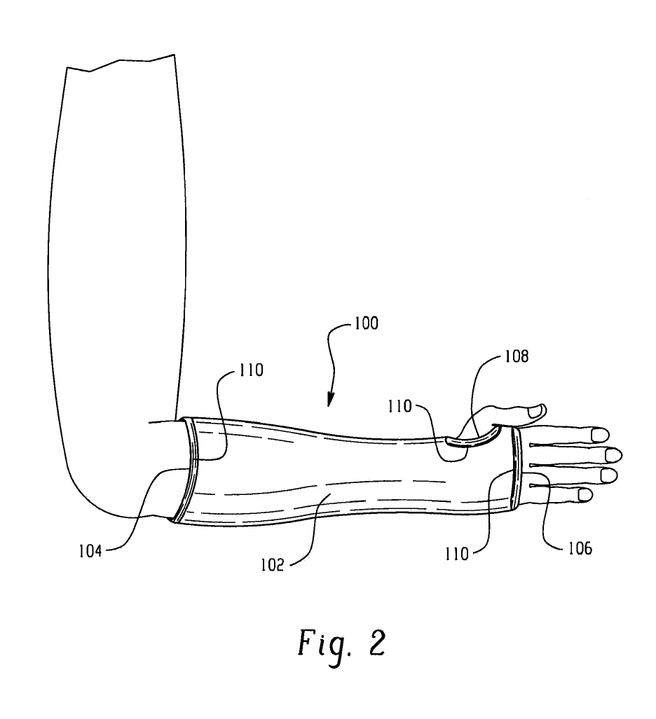

Turning now to FIG. 2, a perspective view of an exemplary embodiment of a cast cover 100 disposed over the cast 10 is shown. As used herein the term “disposed” generally refers to fitting, conforming, surrounding, placing, and the like of the cast cover over the cast. The cast cover 100 is configured to substantially completely cover the cast and padding 110. The cast cover 100 comprises a sleeve 102 configured to slide over the cast surface. The cast cover 100 further comprises a first aperture 104 for permitting the sleeve 102 to slide over the cast, a second aperture 106 configured to permit use of the fingers out the end of the cast, and a third aperture 108 configured to expose the thumb and permit use thereof A comfort band 110 surrounds each of the apertures 104, 106, 108 and is configured to hold the cast cover in place over the cast and provide a comfortable interface between the skin of the wearer and the cast cover.

The cast cover 100 comprises a material which will prevent damage to clothing and other objects, while concealing the cast and protecting the wearer from the often sharp and/or uncomfortable edges of the cast with a conformable, compliant, adjustable fitted material that is soft to the touch. Other considerations for the cast cover material can include, without limitation, durability, softness, breathability, decorative, washability, and the like. In one embodiment, the cast cover 100 is made of a knitted fabric material. In another embodiment, the cast cover 100 can also comprise a stretchable material so as to accommodate casts of various sizes and shapes. Exemplary materials for the cast cover 100 can include, without limitation, cotton, a cotton/polyester blend, nylon, silk, a polyurethane-polyurea copolymer, such as LYCRA®, Spandex, elastane, and the like, or combinations comprising one of the foregoing.

In one embodiment, the cast cover 100 can comprise a single material. In another embodiment, the cast cover 100 can be constructed of more than one material. For example, the sleeve 102 can comprise one material, while the comfort band(s) 110 can comprise a different material. The comfort band 110 is configured to comfortably secure the cast cover over the cast and can be in physical communication with one or more areas of skin on the wearer. As mentioned, the comfort band 110 provides an advantage over existing cast covers that utilize elastic ends. Unlike a narrow elastic band edge of a cast cover that is prone to pinching the skin and irritating it after short use, the comfort bands provide soft, comfortable security to the cast cover on the cast, while also providing longer wearability with little to no discomfort or irritation to the wearer's skin.

In the embodiment of FIG. 2, for instance, the comfort bands 110 are in contact with the wearer's forearm, hand, and thumb. Generally, to secure the cast cover in position and prevent it from sliding off the cast or twisting in position, the comfort band will provide snug fit without overly constricting these areas. The comfort bands, therefore, can have more elasticity than the sleeve. In one embodiment, the comfort band 110 comprises a knitted-in two-ply circumferential portion that is positioned about one of the apertures. The comfort band may be knit into the end of the sleeves about the apertures 104, 106, 108. The comfort band can include two plies formed by extending the knit of the sleeve into a first layer of the band, making a turn at the edge of the comfort band and knitting the second layer formed by the turn to the first (now inside) inside layer. Then, the layers are knit together on the inside of the cast cover at the end of the aperture. For example, the comfort band 110 of knitted-in two-ply material can be knit using a feed of 120 denier LYCRA SOFT that is single covered with 70 denier, 48 filament nylon.

In another embodiment, the comfort band 110 and the sleeve 102 could end at the apertures in a knitted-in welt 110. During manufacture of the comfort band, all of the feeds preferably use the same yarns, such as the 120 denier LYCRA SOFT that is single covered with 70 denier, 48 filament nylon or some other denier and filament nylon, but could also alternate different yarns from one through four feeds.

In still another embodiment, a single rubber yarn (e.g., elastic) may be knitted into the comfort band 110 to ensure that the cast cover is secured about the cast of the wearer. However, the rubber yarn is merely optional, because the denier/fabric used in the comfort band region is believed to be sufficient to provide the needed support, security, and comfort to the cast cover.

The comfort band will have a width suitable for securing the cast cover and will depend on the size of the cast cover, the shape and position of the cast, the material of the sleeve, and the like. In one embodiment, the comfort band has a width of about 0.1 inches to about 3 inches; specifically a width of about 0.25 inches to about 2 inches; and more specifically about 0.5 inches to about 1 inch. When the cast cover comprises more than one comfort band, the different comfort bands can have the same widths or they can have different widths.

Whatever the type of material chosen for the cast cover 100, the cover can come in a wide variety of colors, patterns, designs, and the like. In one embodiment, the cast cover 100 can be a single unitary color. In another the embodiment, the cast cover 100 can comprise multiple colors. For example the sleeve 100 can have one color and the comfort bands 110 can have one or more different colors. In still another example, the cast cover 100 comprises a patterned material. The pattern can cover only the sleeve 102, with the comfort bands 110 having a different pattern or a solid color, or the pattern can cover all of the material of the cast cover 100. The cast cover 100 can also include decoration, such as embellishments that add to the dressiness, design, or attractiveness of the cast cover. For example, the cast cover 100 can further include rhinestones incorporated on the sleeve. As used herein, “incorporated” refers to the manner in which the decoration is attached to the cast cover and is intended to encompass, without limitation, the decoration being adherent, sewn on, velcroed, or the like to the sleeve 102. The rhinestones can be configured to provide any pattern or design on the sleeve, including but not limited to smile face, frown face, heart, Fleur de lis, etc. Other examples of embellishments can include, without limitation, attachable or Velcro-backed accessories such as buttons, pins, sequins, and the like.

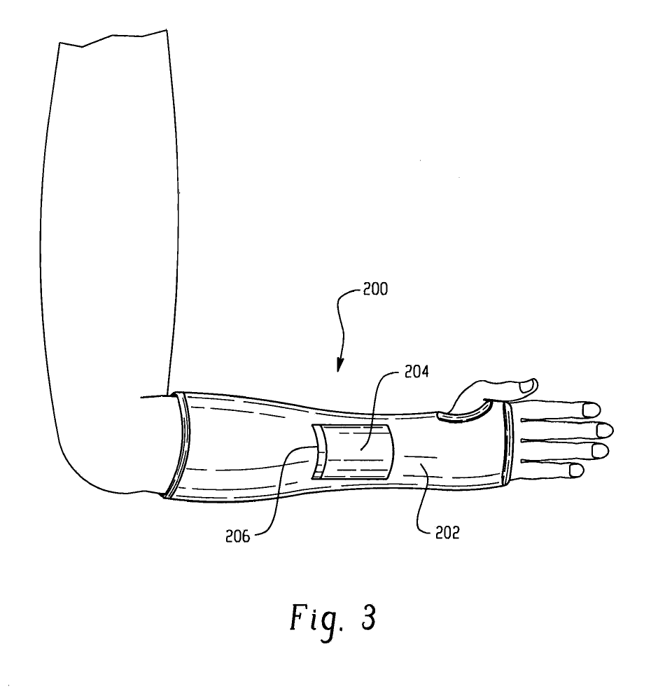

FIG. 3 is a perspective view of another embodiment of a cast cover 200. In this embodiment, a sleeve 202 of the cast cover further comprises a pocket 204. The pocket 204 is secured/attached to the sleeve 202 by bonding, sewing, or other methods known in the textile arts. The pocket 204 can be of any convenient size and shape, and enables the wearer to carry any number of articles. For example, the pocket 204 can carry a cell phone, keys, credit cards, driver's license, passport, change, tissues, and the like. Preferably, such objects would be light in weight, so as not to add to much weight to the cast or over stretch the cover material. In the case of a children's cast cover, the pocket 204 can carry toys, such as, plastic figures, stuffed animals, figurines, toy cars, and the like. To prevent the inadvertent loss of items in this pocket 204, a hook and loop type fastener, zipper, snap, button, or the like, can be disposed near the top 206 of the pocket for closing the pocket. For example, the top of the pocket can comprise hook and loop material sold under the trademark VELCRO, wherein the hook portion of the material is pushed together with the loop portion of the material to close the top of the pocket 204. Similarly, the hook portion of the material is pulled apart from the loop portion of the material to open the top of the pocket 204.

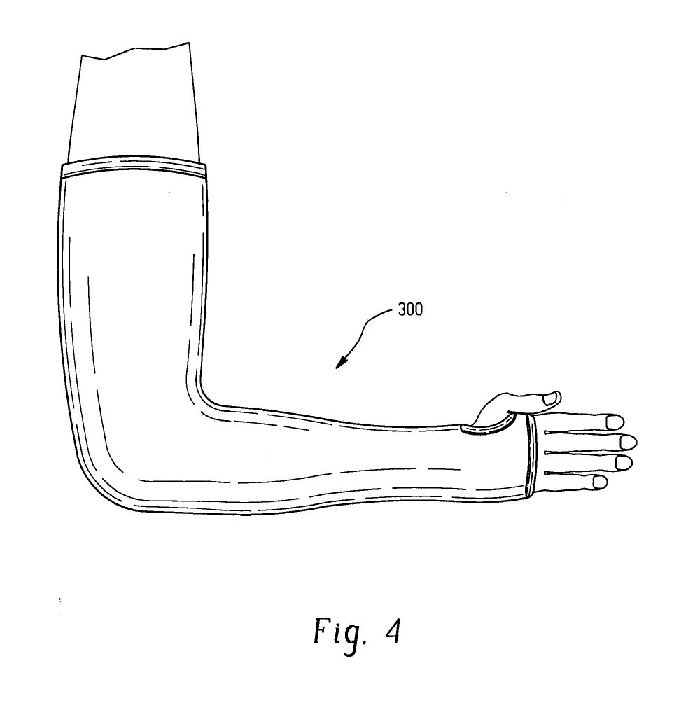

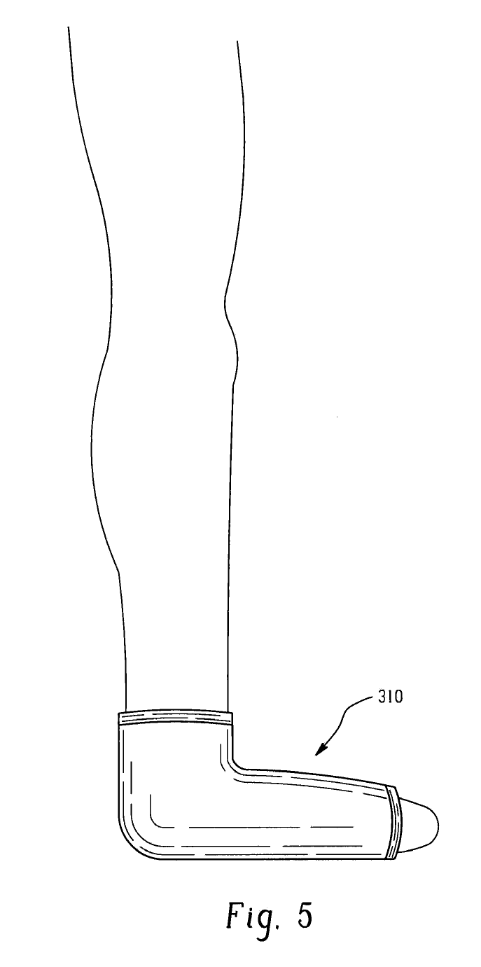

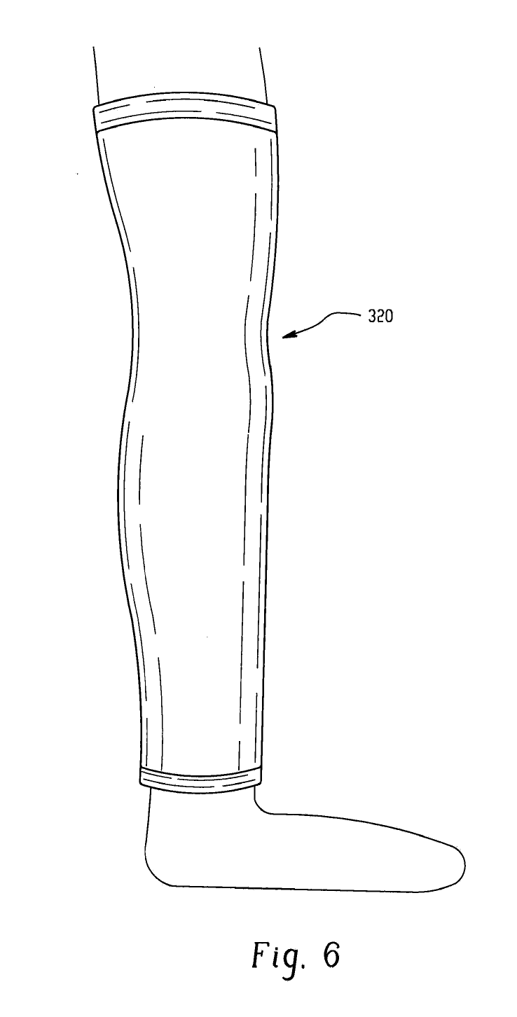

As mentioned, the cast covers described herein can be used to cover any type of cast disposed on any injured portion of the body. FIGS. 4-6 illustrate cast covers for use with a few of the more common types of casts. FIG. 4 is a perspective view of a cast cover 300 on an elbow/arm cast. FIG. 5 is a perspective view of a cast cover 310 on a lower leg/ankle cast. FIG. 6 is a perspective view of a cast cover 320 on a full leg cast. It is to be understood, however, that these figures are merely for illustration, and the cast cover can be envisioned for use with any cast designed to protect an injured portion of the body. Also of great benefit would be a comfort wrap, like the cast cover, but which could be wrapped like an Ace Bandage around the injury and fastened, for example, with Velcro fasteners. The comfort wrap could be used in place of a cast for minor injuries, such as sprains, and the like.

The cast covers as described herein aid in protecting the cast from the elements and helping with cleanliness and hygiene during its life span, while also helping to conceal or improve the aesthetics of the cast. The cast cover can be accompanied by deodorant packets or wafers to be slipped in between the cover and the cast. Moreover, the cast cover can protect a wearer's clothes, skin, and the like from being scratched, pulled, torn, etc. by the sometimes sharp edges and/or surfaces of the cast. The comfort bands of the cast covers described herein provide certain advantages over existing cast covers in that the bands secure the cast cover in its proper place over the cast, without causing discomfort to the wearer that is sometimes caused by other securing methods such as narrow elastic bands. Further, many doctors must now carry multiple colors of casting wraps in an effort to please patients and provide a more desirable cast compared to the off white plaster casts of old. Not only is the number of available colors for these cast types very limited, but a significant amount of a doctor's office space and storage can be consumed with boxes upon boxes of these tapes adding to the doctor's investment and inventory requirements. The cast cover can eliminate this inconvenience, because a doctor can simply keep one color of cast on hand and then offer cast covers in a wide variety of colors, patterns, designs, etc. to appease the patient.

The terminology used herein is for the purpose of describing particular embodiments only and is not intended to be limiting of the invention. Ranges disclosed herein are inclusive and combinable (e.g., ranges of “up to about 25 vol %, or, more specifically, about 5 vol % to about 20 vol %”, is inclusive of the endpoints and all intermediate values of the ranges of “about 5 vol % to about 25 vol %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the colorant(s) includes one or more colorants). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

Unless otherwise defined, all terms (including technical and scientific terms) used herein have the same meaning as commonly understood by one of ordinary skill in the art to which the embodiments of the invention belong. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A cast cover, comprising:
a sleeve configured to be disposed over a cast;
an aperture configured to receive the cast when pulling the sleeve over the cast; and
a comfort band generally disposed about the aperture, wherein the comfort band is configured to be in physical communication with the skin of a cast wearer and covers an edge of the cast.

2. The cast cover of claim 1, wherein the comfort band comprises an elasticity that is greater than an elasticity of the sleeve.

3. The cast cover of claim 2, wherein the comfort band comprises a knitted-in two-ply circumferential portion about the aperture.

4. The cast cover of claim 3, wherein the comfort band has a width of about 0.1 inches to about 3 inches.

5. The cast cover of claim 1, further comprising one or more additional apertures configured to receive one or more appendages that protrude from the cast.

6. The cast cover of claim 5, wherein the one or more additional apertures include a comfort band.

7. The cast cover of claim 1, wherein the sleeve comprises a knitted fabric material.

8. The cast cover of claim 7, wherein the material comprises cotton, cotton-polyester blend, nylon, silk, a polyurethane-polyurea copolymer, spandex, elastane, or a combination comprising at least one of the foregoing.

9. The cast cover of claim 1, wherein the comfort band is free of rubber yarn.

10. The cast cover of claim 1, further comprising a decoration incorporated onto the sleeve.

11. The cast cover of claim 1, wherein the sleeve and/or the comfort band further comprise a knitted-in welt.

12. The cast cover of claim 1, further comprising a pocket disposed on the sleeve.

13. The cast cover of claim 12, wherein the pocket comprises a closure disposed near a top of the pocket, wherein the closure is configured to prevent the inadvertent loss of items disposed within the pocket.

14. A cast cover, comprising:
a sleeve formed of a first material and configured to be disposed over a cast
an aperture configured to receive the cast when pulling the sleeve over the cast; and
at least one comfort band formed of a second material disposed about the aperture, wherein the comfort band is configured to be in physical communication with the skin of a cast wearer and covers an edge of the cast.

15. The cast cover of claim 14, wherein an elasticity of the second material is greater than an elasticity of the first material.

16. The cast cover of claim 14, wherein the comfort band is knit into the an end of the sleeve about the aperture.

17. The cast cover of claim 14, wherein the second material comprises two plies.

18. The cast cover of claim 14, wherein the comfort band is free of rubber yarn.

19. The cast cover of claim 14, wherein the sleeve and/or the comfort band further comprise a knitted-in welt.

20. A method of making a cast cover, comprising:
providing a sleeve configured to be disposed about a cast, wherein the sleeve comprises at least one aperture;
positioning a comfort band about the at least one aperture, wherein the comfort band is configured to be in physical communication with the skin of a cast wearer and covers an edge of the cast, and wherein forming the comfort band, comprises
extending a knit of the sleeve into a first layer of the comfort band;
making a turn at an edge of the comfort band;
knitting a second layer formed by the turn o the first layer; and
knitting the first and the second layer together on an inside surface of the cast cover near the aperture.
